(19) United States
(12) Patent Application Publication
(10) Pub. No.: US 2017/0251936 A1
(43) Pub. Date: Sep. 7, 2017

(54) BIOLOGICAL INFORMATION MEASUREMENT APPARATUS AND BIOLOGICAL INFORMATION MEASUREMENT METHOD

(71) Applicant: SEIKO EPSON CORPORATION, Tokyo (JP)

(72) Inventors: Ayae SAWADO, Kai-shi (JP); Yuta MACHIDA, Chino-shi (JP)

(73) Assignee: SEIKO EPSON CORPORATION, Tokyo (JP)

(21) Appl. No.: 15/438,031

(22) Filed: Feb. 21, 2017

(30) Foreign Application Priority Data
Mar. 4, 2016 (JP) 2016-042291

(51) Int. Cl. A61B 5/021; A61B 8/06; A61B 5/00; A61B 8/08; A61B 5/02; A61B 5/026; A61B 8/04

(52) U.S. Cl. CPC A61B 5/02125; A61B 5/0261; A61B 8/06; A61B 8/04; A61B 8/5223; A61B 5/02007; A61B 5/681; A61B 5/7278

(57) ABSTRACT

A biological information measurement apparatus includes an irradiation unit configured to irradiate a living body with light or sound waves as measurement waves; a detection unit configured to detect the measurement waves having passed through the inside of the living body; and a computational unit configured to obtain a change over time in blood flow rate and a change over time in blood vessel cross-sectional area based on a detection result from the detection unit, and to obtain a pulse wave propagation velocity from the change over time in blood flow rate or the change over time in blood vessel cross-sectional area.

### BACKGROUND

1. Technical Field

The present invention relates to technology for measuring biological information.

2. Related Art

JP-A-2011-24676 discloses a pulse wave propagation velocity calculation apparatus, pulse wave sensors of which are mounted on at least two measurement sites such as a finger and a wrist, and which calculates a pulse wave propagation velocity using pulse waveforms detected by the pulse wave sensors.

The pulse wave propagation velocity calculation apparatus disclosed in JP-A-2011-24676 calculates a pulse wave propagation velocity from a time difference in propagation of pulse waves between the measurement sites. For this reason, if the distance between the measurement sites is short, the accuracy of calculation of a pulse wave propagation velocity decreases. As a result, it is difficult to reduce the size of the apparatus.

### SUMMARY

An advantage of some aspects of the invention is to reduce the size of an apparatus that measures a pulse wave propagation velocity.

A biological information measurement apparatus according to a first aspect of the invention includes: an irradiation unit configured to irradiate a living body with light or sound waves as measurement waves; a detection unit configured to detect the measurement waves having passed through the inside of the living body; and a computational unit configured to obtain a change over time in blood flow rate and a change over time in blood vessel cross-sectional area based on a detection result from the detection unit, and to obtain a pulse wave propagation velocity from the change over time in blood flow rate or the change over time in blood vessel cross-sectional area.

In this configuration, the biological information measurement apparatus obtains a pulse wave propagation velocity from a change over time in blood flow rate and a change over time in blood vessel cross-sectional area. Therefore, it is not necessary for a subject to wear sensors on multiple measurement sites such as a finger and a wrist, the number of measurement sites may be one, and the number of sensors (an irradiation unit and a detection unit) for measurement may be one. As a result, it is possible to reduce the size of the biological information measurement apparatus.

In the biological information measurement apparatus according to the first aspect of the invention, the computational unit may obtain a blood pressure or the degree of arteriosclerosis using the pulse wave propagation velocity (second aspect). In this case, the biological information measurement apparatus is capable of obtaining a blood pressure or the degree of arteriosclerosis in addition to a pulse wave propagation velocity.

In the biological information measurement apparatus according to the first or second aspect of the invention, the measurement waves may be laser beams, the detection unit may generate an optical beat signal representing changes over time in light receiving intensity and frequency of the laser beams having passed through the inside of the living body, and the computational unit may obtain the change over time in blood flow rate and the change over time in blood vessel cross-sectional area from the optical beat signal generated by the detection unit (third aspect). In this case, the biological information measurement apparatus is capable of obtaining both a change over time in blood flow rate and a change over time in blood vessel cross-sectional area, which are required to obtain a pulse wave propagation velocity, via measurement by a laser Doppler flowmetry method (hereinafter, referred to as an LDF method) using laser beams.

In the biological information measurement apparatus according to the third aspect of the invention, the computational unit may obtain a change over time in the full power of the optical beat signal (fourth aspect). The change over time in the full power of the optical beat signal is equivalent to a plethysmogram. Accordingly, the biological information measurement apparatus of the fourth aspect is capable of obtaining a plethysmogram in addition to a pulse wave propagation velocity via measurement by an LDF method using laser beams.

In the biological information measurement apparatus according to the first or second aspect of the invention, the measurement waves may be non-laser beams, the detection unit may generate a received light signal representing a change over time in light receiving intensity of the non-laser beams having passed through the inside of the living body, and the computational unit may obtain the change over time in blood flow rate and the change over time in blood vessel cross-sectional area from the received light signal generated by the detection unit (fifth aspect). In this case, the biological information measurement apparatus is capable of obtaining both a change over time in blood flow rate and a change over time in blood vessel cross-sectional area, which are required to obtain a pulse wave propagation velocity, via measurement using non-laser beams.

In the biological information measurement apparatus according to the first or second aspect of the invention, the irradiation unit may include a first irradiation unit configured to irradiate the living body with laser beams, and a second irradiation unit configured to irradiate the living body with non-laser beams, the detection unit may include a first detection unit configured to detect the laser beams having passed through the inside of the living body, and a second detection unit configured to detect the non-laser beams having passed through the inside of the living body, and the computational unit may obtain a change over time in blood flow rate based on a detection result from the first detection unit, and obtains a change over time in blood vessel cross-sectional area based on a detection result from the second detection unit (sixth aspect). In this case, the biological information measurement apparatus obtains a change over time in blood flow rate via measurement using laser beams, and obtains a change over time in blood vessel cross-sectional area via measurement using non-laser beams. Accordingly, it is possible to accurately obtain the change over time in blood flow rate and the change over time in blood vessel cross-sectional area. As a result, it is possible to improve the accuracy of computation of a pulse wave propagation velocity.

In the biological information measurement apparatus according to the first or second aspect of the invention, the irradiation unit may include a first irradiation unit configured to irradiate the living body with laser beams, and a second irradiation unit configured to irradiate the living body with non-laser beams, the detection unit may detect the laser beams and the non-laser beams having passed through the inside of the living body, and the computational unit may obtain a change over time in blood flow rate based on a result of detecting the laser beams via the detection unit, and obtains a change over time in blood vessel cross-sectional area based on a result of detecting the non-laser beams via the detection unit (seventh aspect). In this case, the number of detection units may be one, and it is not necessary to separately provide a detection unit for detecting laser beams and a detection unit for detecting non-laser beams. As a result, it is possible to further simplify the configuration of the biological information measurement apparatus and to further reduce the size of the biological information measurement apparatus than those of the biological information measurement apparatus of the sixth aspect of the invention.

In the biological information measurement apparatus according to the sixth or seventh aspect of the invention, a site of the living body, from which a change over time in blood flow rate is obtained by irradiating the site with laser beams, may be the same as a site of the living body from which a change over time in blood vessel cross-sectional area is obtained by irradiating the site with non-laser beams (eighth aspect). In this case, it is possible to obtain a pulse wave propagation velocity from the change over time in blood flow rate and the change over time in blood vessel cross-sectional area which are obtained from the same site. As a result, it is possible to accurately obtain a pulse wave propagation velocity of a local site (measurement site). Since a site from which a change over time in blood flow rate is obtained by irradiating the site with laser beams is the same as a site from which a change over time in blood vessel cross-sectional area is obtained by irradiating the site with non-laser beams, it is possible to further reduce the size of the biological information measurement apparatus than that of a biological information measurement apparatus in a case where both the sites are different.

A biological information measurement method according to a ninth aspect of the invention includes: irradiating a living body with light or sound waves as measurement waves via a biological information measurement apparatus; detecting the measurement waves, which have passed through the inside of the living body, via the biological information measurement apparatus; obtaining a change over time in blood flow rate and a change over time in blood vessel cross-sectional area based on a detection result via the biological information measurement apparatus; and obtaining a pulse wave propagation velocity obtaining a pulse wave propagation velocity from the change over time in blood flow rate and the change over time in blood vessel cross-sectional area. According to this aspect of the invention, it is possible to obtain the same effects as those of the biological information measurement apparatus of the first aspect of the invention.

### BRIEF DESCRIPTION OF THE DRAWINGS

The invention will be described with reference to the accompanying drawings, wherein like numbers reference like elements.

FIG. 1 is view illustrating a state in which a subject wears a biological information measurement apparatus of a first embodiment on a wrist.

FIG. 2 is a front view of the biological information measurement apparatus.

FIG. 3 is a rear view of the biological information measurement apparatus.

FIG. 4 is a block diagram of the biological information measurement apparatus.

FIG. 5 is a schematic view illustrating a principle of measuring biological information via an LDF method.

FIG. 6 is a flowchart illustrating a biological information measurement process of the first embodiment.

FIG. 7 is a block diagram illustrating a biological information measurement apparatus of a second embodiment.

FIG. 8 is a flowchart illustrating a biological information measurement process of the second embodiment.

FIG. 9 is a graph illustrating a plethysmogram and the blood flow waveform.

FIG. 10 is a graph illustrating a blood pressure.

FIG. 11 is a block diagram of a biological information measurement apparatus of a third embodiment.

FIG. 12 is a flowchart illustrating a biological information measurement process of the third embodiment.

FIG. 13 is a block diagram of a biological information measurement apparatus of a fourth embodiment.

FIG. 14 is a view illustrating the disposition of optical sensors.

FIG. 15 is a flowchart illustrating a biological information measurement process of the fourth embodiment.

FIG. 16 is a view illustrating the configuration of a biological information measurement module of a modification example.

FIG. 17 is a schematic view illustrating a principle of measuring biological information using an ultrasonic sensor of the modification example.

### DESCRIPTION OF EXEMPLARY EMBODIMENTS

Hereinafter, embodiments of the invention will be described with reference to the accompanying drawings.

### First Embodiment

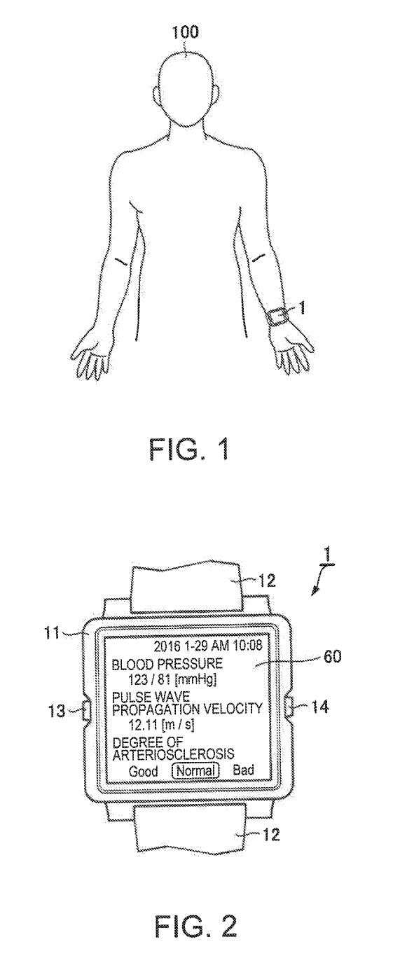

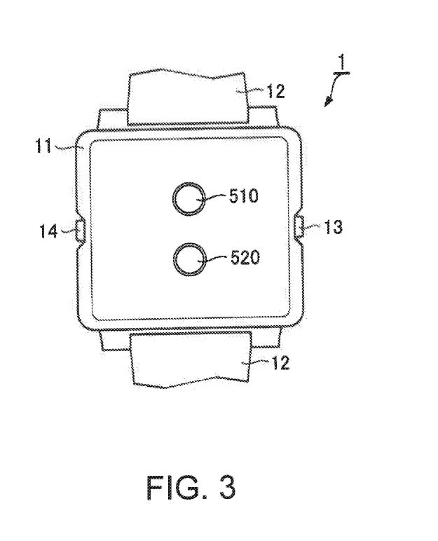

FIG. 1 is a view illustrating a state in which a subject 100 wears a biological information measurement apparatus 1 of a first embodiment of the invention on a wrist. FIG. 2 is a front view of the biological information measurement apparatus 1, and FIG. 3 is a rear view of the biological information measurement apparatus 1. The biological information measurement apparatus 1 is a measurement device that measures biological information regarding the subject (living body) 100 in a non-invasive manner. As illustrated in FIG. 1, the biological information measurement apparatus 1 is a wrist-watch type wearable device which the subject 100 wears on the wrist. The biological information measurement apparatus 1 is an optical blood pressure meter, and is capable of measuring a blood pressure or the degree of arteriosclerosis as biological information in addition to a pulse wave propagation velocity.

As illustrated in FIGS. 2 and 3, the biological information measurement apparatus 1 includes a main body portion 11 and a belt 12. The belt 12 is wrapped around the wrist of the subject 100. As illustrated in FIG. 2, a display unit 60 is provided on a front surface (surface opposite to a surface in contact with an epidermis of the wrist of the subject 100) of the main body portion 11. As illustrated in FIG. 2, the display unit 60 displays biological information (blood pressure, pulse wave propagation velocity, the degree of arteriosclerosis, and the like) regarding the subject 100 which are measured by the biological information measurement apparatus 1. Two operation buttons 13 and 14 are provided on lateral surfaces of the main body portion 11. The subject 100 can issue an instruction to start measurement of biological information, or perform various settings related to measurement of biological information by operating the operation buttons 13 and 14. As illustrated in FIG. 3, a rear surface (surface in contact with the epidermis of the wrist of the subject 100) of the main body portion 11 is provided with a laser beam emitting unit 510 which is an example of an irradiation unit, and a laser beam receiving unit 520 which is an example of a detection unit.

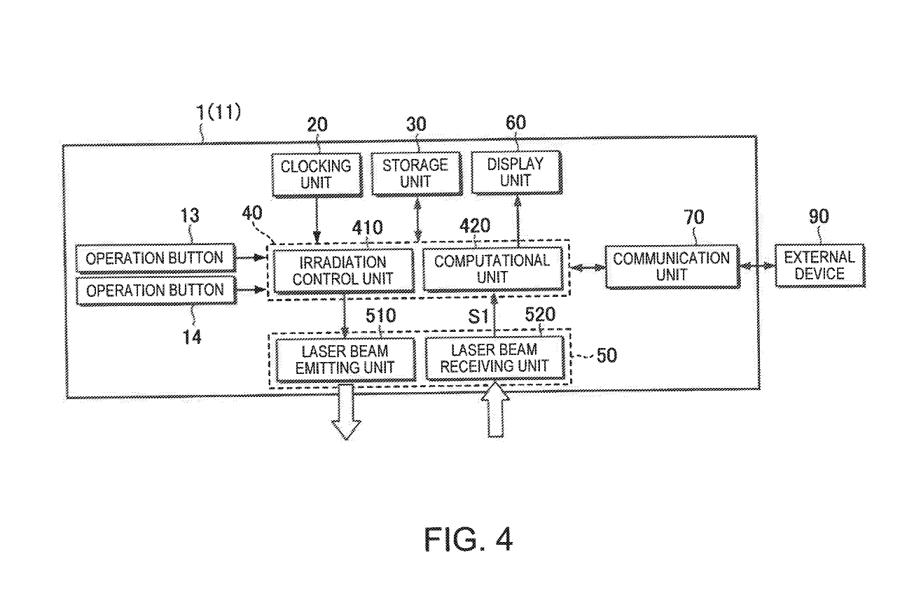

FIG. 4 is a block diagram illustrating the inner configuration of the biological information measurement apparatus 1. The biological information measurement apparatus 1 includes the operation buttons 13 and 14; a clocking unit 20; a storage unit 30; a control unit 40; an optical sensor 50; the display unit 60; and a communication unit 70. The operation buttons 13 and 14 output operation signals to the control unit 40. The clocking unit 20 includes an oscillation circuit or a frequency dividing circuit, and measures a time including year, month, day, hour, minute, and second. The storage unit 30 includes a non-volatile semiconductor memory, and stores a program executed by the control unit 40, and various data used by the control unit 40.

The control unit 40 is a computational processing device such as a central processing unit (CPU) or a field-programmable gate array (FPGA), and controls the entirety of the biological information measurement apparatus 1. The control unit 40 executes various processes related to measurement of biological information by executing the program stored in the storage unit 30. The control unit 40 includes an irradiation control unit 410 and a computational unit 420. The irradiation control unit 410 controls irradiation of laser beams performed by the laser beam emitting unit 510. The computational unit 420 obtains biological information regarding the subject 100 by computing a received light signal S1 output from the laser beam receiving unit 520. The biological information obtained by the computational unit 420 includes the degree of arteriosclerosis, a pulse wave propagation velocity, and a blood pressure.

It is possible to adopt a configuration in which functions of the control unit 40 are dispersed into multiple integrated circuits, or a configuration in which a portion of functions or the entire functions of the control unit 40 are realized by a dedicated electronic circuit. In FIG. 4, the control unit 40 and the storage unit 30 are illustrated as separate elements. Alternatively, the control unit 40 with the built-in storage unit 30 can be realized by an application specific integrated circuit (ASIC) or the like.

The optical sensor 50 includes the laser beam emitting unit 510 and the laser beam receiving unit 520. The laser beam emitting unit 510 includes a semiconductor laser, a laser drive circuit, and the like. The laser beam emitting unit 510 is controlled by the irradiation control unit 410 such that the laser beam emitting unit 510 irradiates the wrist of the subject 100 with laser beams which are an example of a measurement wave. Laser beams irradiated by the laser beam emitting unit 510 are rectilinear beams which are emitted via resonance of resonator and are coherent in a narrow band. For example, laser beams irradiated by the laser beam emitting unit 510 have a wavelength of 850 nm.

The laser beam receiving unit 520 includes a light receiving element such as a photo diode; an amplifier; an A-to-D converter; and the like. The light receiving element has narrow band-pass characteristics corresponding to the wavelength of laser beams irradiated by the laser beam emitting unit 510, selectively transmits only light of the corresponding wavelength region, and blocks light (sunlight, white light, and the like) of other wavelength regions. The laser beam receiving unit 520 receives laser beams having passed through the inside of the living body of the subject 100 via the light receiving element, and generates and outputs the received light signal S1, which indicates changes over time in light receiving intensity and frequency of the laser beams, to the computational unit 420.

The display unit 60 is a liquid crystal display or an organic electroluminescence (EL) display. The display unit 60 displays biological information and the like regarding the subject 100 which are output from the computational unit 420 (refer to FIG. 2). The communication unit 70 controls communication with an external device 90 such as a personal computer or a smart phone. The communication unit 70 communicates with the external device 90 via radio communication such as Bluetooth (registered trademark), Wi-Fi, or infrared communication. The communication unit 70 is capable of communicating with the external device 90 via wire communication using a communication cable.

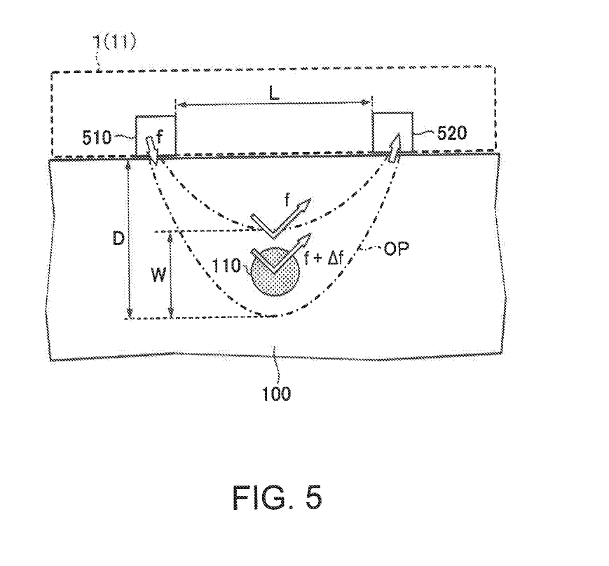

FIG. 5 is a schematic view illustrating a principle of measuring biological information via an LDF method. The rear surface (a light emitting surface of the laser beam emitting unit 510 and a light receiving surface of the laser beam receiving unit 520) of the main body portion 11 is in close contact with the epidermis of the wrist of the subject 100. Laser beams irradiated by the laser beam emitting unit 510 transmit through the epidermis, and are incident into the wrist of the subject 100 (into the living body). The laser beams incident into the living body spreads through biological tissues while being repeatedly scattered and reflected, and a portion of the laser beams reaches the laser beam receiving unit 520, and is received by the light receiving element.

If the frequency of the laser beams irradiated by the laser beam emitting unit 510 is assumed to be f, the frequency of laser beams scattered by stationary tissues such as epidermises, coria, and subcutaneous tissues does not change. In contrast, laser beams scattered by blood cells such as red blood cells flowing through a blood vessel 110 are subjected to a very small wavelength shift Δf corresponding to the flow velocity of the blood cells, and light intensity changes in correspondence with the amount of the flowing blood cells. Accordingly, scattered light (laser beam) having the frequency f caused by the stationary tissues interferes with scattered light (laser beam) having a frequency f+Δf including a Doppler shift caused by the blood cells.

For this reason, optical beats having a difference frequency Δf occur, and the received light signal S1 generated by the laser beam receiving unit 520 has a waveform in which an intensity-modulated signal having the optical beat frequency Δf is superimposed on a DC signal. Since the received light signal S1 has a waveform in which the velocity (frequency) of fluctuation and the magnitude (amplitude) of light intensity correspond to the flow velocity and the amount of blood cells, it is possible to obtain a blood flow rate, a blood volume, and the like by computing the received light signal S1. As being apparent from the aforementioned description, the received light signal S1 is an optical beat signal indicating changes over time in light receiving intensity and frequency of laser beams having passed through the inside of the living body of the subject 100.

If a high distribution frequency region of propagation paths of laser beams having reached the laser beam receiving unit 520 is schematically illustrated, the high distribution frequency region has a banana shape (region interposed between two arcs) illustrated by alternate long and short dash lines in FIG. 5. A width W of a passing region OP in a depth direction is widest in the vicinity of the center thereof. A measurement depth (depth from the epidermis which can be reached by laser beams irradiated by the laser beam emitting unit 510) D is shallower as a separation distance L between the laser beam emitting unit 510 and the laser beam receiving unit 520 is decreased. The measurement depth D is deeper as the separation distance L is increased. Accordingly, the separation distance L between the laser beam emitting unit 510 and the laser beam receiving unit 520, or both the laser beam emitting unit 510 and the laser beam receiving unit 520 in the main body portion 11 are positioned such that the blood vessel (for example, an artery) 110 which is a measurement target is placed in a portion of the passing region OP, which has the widest width W in the depth direction.

The passing region OP illustrated in FIG. 5 is a mere image for the sake of convenience. Actual propagation paths of the laser beams having reached the laser beam receiving unit 520 are not limited to the passing region OP, and various paths can be obtained. In FIG. 5, for the sake of convenience, only one blood vessel 110 is illustrated, and actually, measurement targets are all blood vessels which are present on the propagation paths of the laser beams having reached the laser beam receiving unit 520. Accordingly, a blood flow rate and a blood volume obtained by computing the received light signal S1 are a tissue blood flow rate and a tissue blood volume of biological tissues in the reach range of laser beams received by the laser beam receiving unit 520.

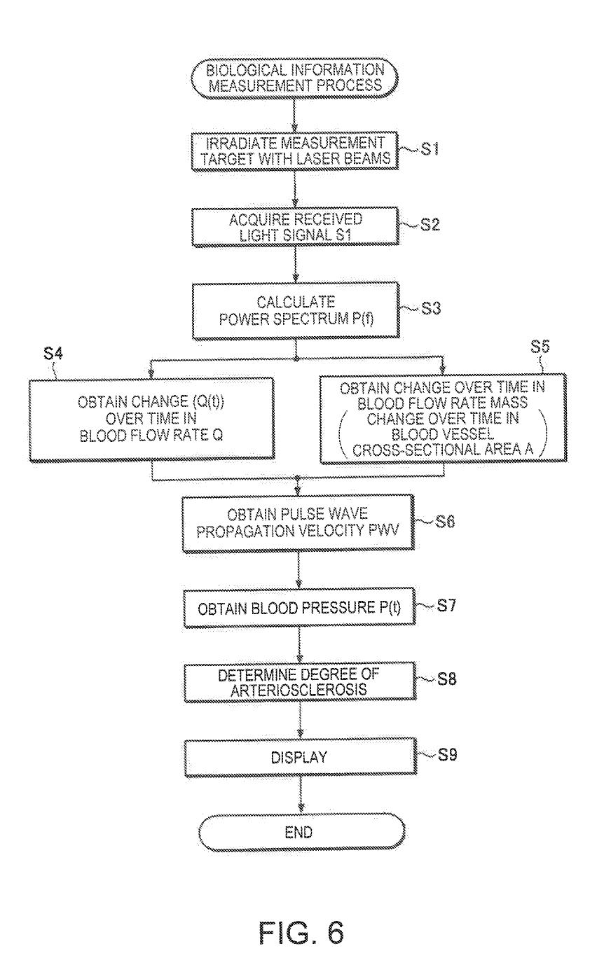

FIG. 6 is a flowchart illustrating a biological information measurement process of the first embodiment. The control unit 40 executes the process illustrated in FIG. 6 whenever a predetermined length of time has elapsed, for example, every five minutes. The process illustrated in FIG. 6 may be executed when the subject 100 issues an instruction to start measurement by operating the operation buttons 13 and 14, or when a clocking time set in advance by the clocking unit 20 reaches a measurement start time.

If the process illustrated in FIG. 6 is started, first, the irradiation control unit 410 of the control unit 40 controls the laser beam emitting unit 510 such that irradiation of laser beams is started (Step S1). Accordingly, the wrist of the subject 100 is irradiated with the laser beams, and the laser beam receiving unit 520 receives laser beams having passed through the inside of the living body of the subject 100, and outputs the received light signal S1 in correspondence with the received laser beams. Subsequently, the computational unit 420 of the control unit 40 acquires the received light signal S1 output from the laser beam receiving unit 520 (Step S2). The computational unit 420 calculates a power spectrum P (f) by performing a frequency analysis process on the acquired received light signal (optical beat signal) S1 via fast Fourier transform (FFT) (Step S3).

Subsequently, the computational unit 420 obtains a change over time in blood flow rate Q from Expression 1 using the calculated power spectrum P(f) (Step S4).

$$Q = \frac{{K}_{1} {\int }_{{f}_{1}}^{{f}_{2}} f \cdot P ( f ) df }{〈 {I}^{2} 〉} \qquad [ Expression 1 ]$$

K₁ represents a proportion constant, f₁ and f₂ represent cutoff frequencies, f represents the frequency of laser beams irradiated by the laser beam emitting unit 510, and <I²> represents the full power of the received light signal S1.

That is, in Step S4, the computational unit 420 calculates the blood flow rate Q by weighting the calculated power spectrum P(f) by the frequency f (f·P(f)), obtaining a primary moment by integrating the resultant in a cutoff frequency range of f₁ to f₂, multiplying the primary moment by the proportion constant K₁, and then normalizing the resultant by the full power <I²> of the received light signal S1 in order for the resultant to be independent of a difference between light receiving intensities of the laser beams. The computational unit 420 calculates the blood flow rate Q for a predetermined period, for examples, for 20 milliseconds.

In parallel with Step S4, the computational unit 420 obtains a change over time in blood volume MASS from Expression 2 using the power spectrum P(f) calculated in Step S3 (Step S5).

$$MASS = \frac{{K}_{2} {\int }_{{f}_{1}}^{{f}_{2}} P ( f ) df}{〈 {I}^{2} 〉} \qquad [ Expression 2 ]$$

K₂ represents a proportion constant.

That is, in Step S5, the computational unit 420 calculates the blood volume MASS by obtaining a primary moment by integrating the calculated power spectrum P(f) in a cutoff frequency range of f₁ to f₂, multiplying the primary moment by the proportion constant K₂, and then normalizing the resultant by the full power <I²> of the received light signal S1 in order for the resultant to be independent of a difference between light receiving intensities of the laser beams. The computational unit 420 calculates the blood volume MASS for a predetermined period, for examples, for 20 milliseconds. A change over time in the blood volume MASS obtained in this manner is equivalent to a change over time in blood vessel cross-sectional area A.

If the calculation period for the blood flow rate Q or the blood vessel cross-sectional area A (blood volume MASS) is sufficiently smaller than that of one beat of a pulse wave, the calculation period can be determined to be an arbitrary length of time. After the computational unit 420 calculates the blood flow rate Q or the blood vessel cross-sectional area A every 1 kHz, the computational unit 420 may smooth the calculated blood flow rate Q or the calculated blood vessel cross-sectional area A in every period of approximately 50 Hz.

Subsequently, the computational unit 420 obtains a pulse wave propagation velocity PWV from Expression 3 using the change over time in the blood flow rate Q obtained in Step S4 and the change over time in the blood vessel cross-sectional area A obtained in Step S5 (Step S6).

$$PWV = \frac{dQ}{dA} \qquad [ Expression 3 ]$$

Subsequently, the computational unit 420 obtains a blood pressure from Expression 4 using the change (A(t)) over time in the blood vessel cross-sectional area A obtained in Step S5 in addition to the pulse wave propagation velocity PWV obtained in Step S6 (Step S7). In Step S7, a change over time in blood pressure represented by P (t) may be obtained as a blood pressure, or the maximum blood pressure (systolic blood pressure) and the minimum blood pressure (diastolic blood pressure) may be obtained as a blood pressure.

$$P ( t ) = p + {ρ PWV}^{2} \frac{A ( t ) - a}{a} \qquad [ Expression 4 ]$$

p represents an average arterial blood pressure, p represents the mass density (fixed value) of blood, and a represents the average of the blood vessel cross-sectional area over time.

Subsequently, the computational unit 420 determines the degree of arteriosclerosis using the pulse wave propagation velocity PWV obtained in Step S6 (Step S8). As illustrated in FIG. 2, the degree of arteriosclerosis can be represented by three levels of indicators such as “good”, “normal”, and “bad”. An artery is harder as the pulse wave propagation velocity PWV increases, and is softer as the pulse wave propagation velocity PWV decreases. Accordingly, the storage unit 30 stores a data table defining the numerical ranges of the pulse wave propagation velocity PWV for “good”, “normal”, and “bad”, and determines the degree of arteriosclerosis from the value of the pulse wave propagation velocity PWV, which is obtained in Step S6, with reference to the data table. The computational unit 420 may determine the degree of arteriosclerosis while taking the gender or age of the subject 100 into consideration in addition to the value of the pulse wave propagation velocity PWV.

Thereafter, the control unit 40 outputs the pulse wave propagation velocity PWV obtained in Step S6, the blood pressure (for example, the maximum blood pressure and the minimum blood pressure) obtained in Step S7, and the degree of arteriosclerosis obtained in Step S8 to the display unit 60 together with a command instructing display (Step S9), and ends the biological information measurement process. Accordingly, as illustrated in FIG. 2, the display unit 60 displays the blood pressure and the degree of arteriosclerosis in addition to the pulse wave propagation velocity PWV.

As described above, in the embodiment, the biological information measurement apparatus 1 obtains the pulse wave propagation velocity PWV from a change over time in the blood flow rate Q and a change over time in the blood vessel cross-sectional area A. Therefore, it is not necessary for the subject 100 to wear the optical sensors 50 on multiple measurement sites such as a finger and a wrist, the number of measurement sites may be one, and the number of optical sensors 50 (the laser beam emitting unit 510 and the laser beam receiving unit 520) for measurement may be one. As a result, it is possible to reduce the size of the biological information measurement apparatus 1.

In the embodiment, the biological information measurement apparatus 1 is capable of obtaining both a change over time in the blood flow rate Q and a change over time in the blood vessel cross-sectional area A, which are required to obtain the pulse wave propagation velocity PWV, via measurement by an LDF method using laser beams. The biological information measurement apparatus 1 is capable of obtaining a blood pressure or the degree of arteriosclerosis in addition to a pulse wave propagation velocity PWV as biological information regarding the subject 100, and is capable of continuously measuring the biological information items over a long period of time in a non-invasive and non-pressure manner.

### Second Embodiment

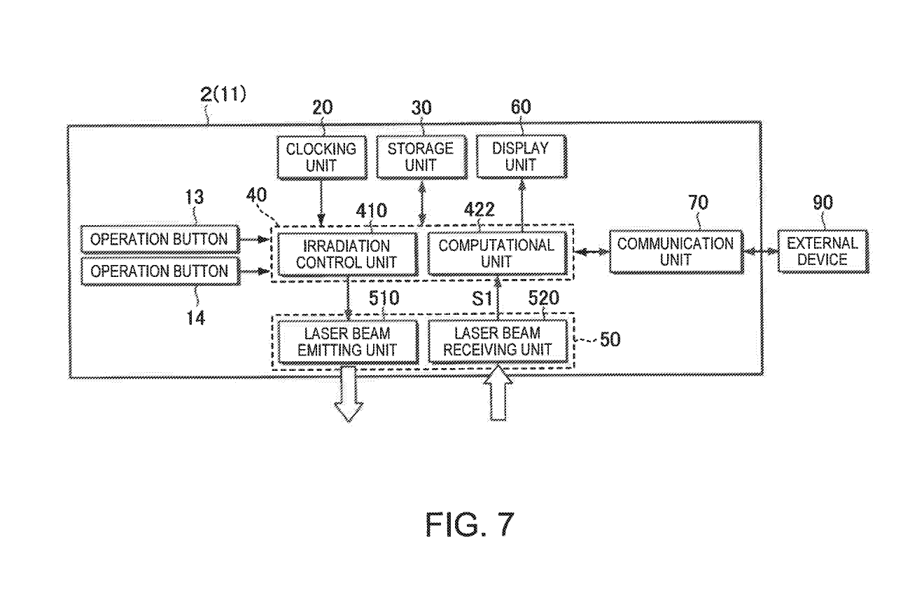

FIG. 7 is a block diagram illustrating the inner configuration of a biological information measurement apparatus 2 of a second embodiment of the invention. In the embodiment, the reference signs used in the first embodiment are assigned to elements common to the first embodiment, and description thereof will be suitably omitted. The biological information measurement apparatus 2 of the second embodiment obtains a “change over time in the blood vessel cross-sectional area A”, which is used to calculate the pulse wave propagation velocity PWV, by a method different from the technique described in the first embodiment. The biological information measurement apparatus 2 of the second embodiment is capable of measuring a plethysmogram as biological information regarding the subject 100. Other portions of the biological information measurement apparatus 2 are the same as those of the biological information measurement apparatus 1 of the first embodiment apart from the aforementioned two points. The difference between the biological information measurement apparatus 2 illustrated in FIG. 7 and the biological information measurement apparatus 1 illustrated in FIG. 4 is that the biological information measurement apparatus 2 includes a computational unit 422.

Accordingly, the laser beam emitting unit 510 of the biological information measurement apparatus 2 of the embodiment also irradiates the wrist of the subject 100 with laser beams. The laser beam receiving unit 520 receives laser beams having passed through the inside of the living body of the subject 100, and generates and outputs the received light signal S1, which is an optical beat signal, to the computational unit 422.

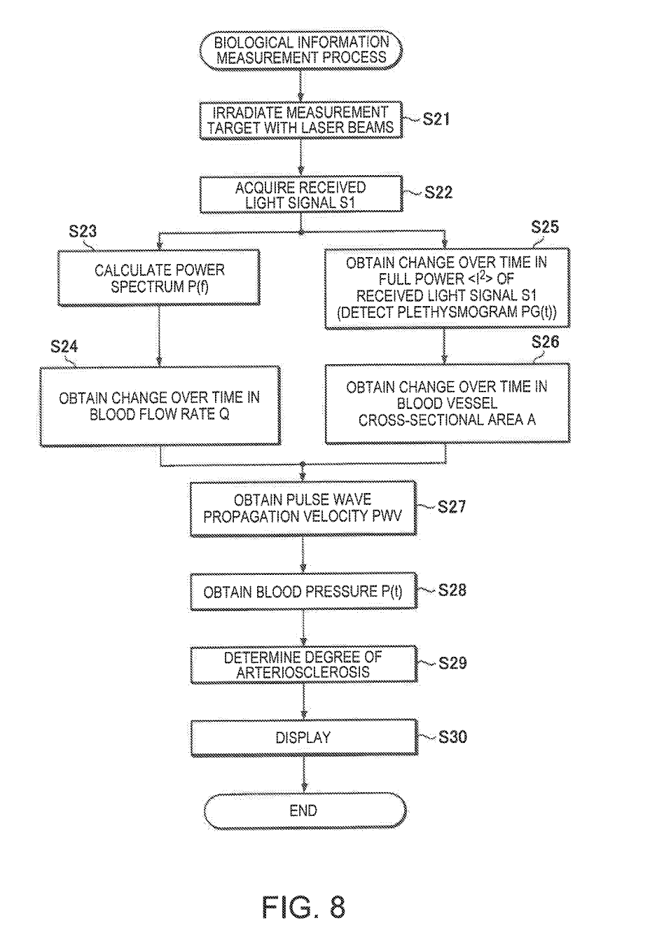

FIG. 8 is a flowchart illustrating a biological information measurement process of the second embodiment. The execution of the process illustrated in FIG. 8 is triggered by the control unit 40 in the same manner as in the process of the first embodiment illustrated in FIG. 6. If the process illustrated in FIG. 8 is started, first, the irradiation control unit 410 of the control unit 40 controls the laser beam emitting unit 510 such that irradiation of laser beams is started (Step S21). The computational unit 422 of the control unit 40 acquires the received light signal S1 output from the laser beam receiving unit 520 (Step S22).

Subsequently, the computational unit 422 calculates the power spectrum P(f) by performing a frequency analysis process on the acquired received light signal (optical beat signal) S1 via fast Fourier transform (FFT) (Step S23). The computational unit 422 obtains a change over time in the blood flow rate Q from Expression 1 described in the first embodiment using the calculated power spectrum P (f) (Step S24). Steps S21 to S24 are the same as Steps S1 to S4 described in the first embodiment.

In parallel with Steps S23 and S24, the computational unit 422 performs a step of detecting a plethysmogram (Step S25) and a step of obtaining a change over time in the blood vessel cross-sectional area A (Step S26). If the step of detecting a plethysmogram is first described, as described in the first embodiment, not only laser beams scattered by blood cells such as red blood cells flowing through the blood vessel 110 are subjected to a Doppler shift corresponding to the flow velocity of the blood cells, but also light intensity changes in correspondence with the amount of the flowing blood cells.

That is, a portion of laser beams with which the inside of the living body is irradiated are absorbed by blood cells (mainly, hemoglobins) flowing through the blood vessel 110. The blood vessel 110 repeatedly expands and contracts in the same period as that of the heartbeat. Accordingly, the amount of blood cells inside of the blood vessel 110 during expansion is different from that during contraction, and thus, intensities of laser beams received by the laser beam receiving unit 520 vary periodically in correspondence with pulsations of the blood vessel 110, and variation components are included in the received light signal S1.

When calculating the power spectrum P(f) in Step S23, the computational unit 422 divides the received light signal S1 into multiple sections having a predetermined length of time, for example, 20 milliseconds, and performs fast Fourier transform for each divided section. The computational unit 422 calculates the full power <I²> of the received light signal S1 for each divided section, for which fast Fourier transform is performed, from Expression 5. Accordingly, the full power <I²> of the received light signal S1 is calculated every 20 milliseconds. As a result, a change over time in the full power <I²> of the received light signal S1 is obtained (Step S25).

$$〈 {I}^{2} 〉 = \frac{1}{t} {\int }_{0}^{t} {I}^{2} ( t ) dt \qquad [ Expression 5 ]$$

I represents light receiving intensities of laser beams received by the light receiving element.

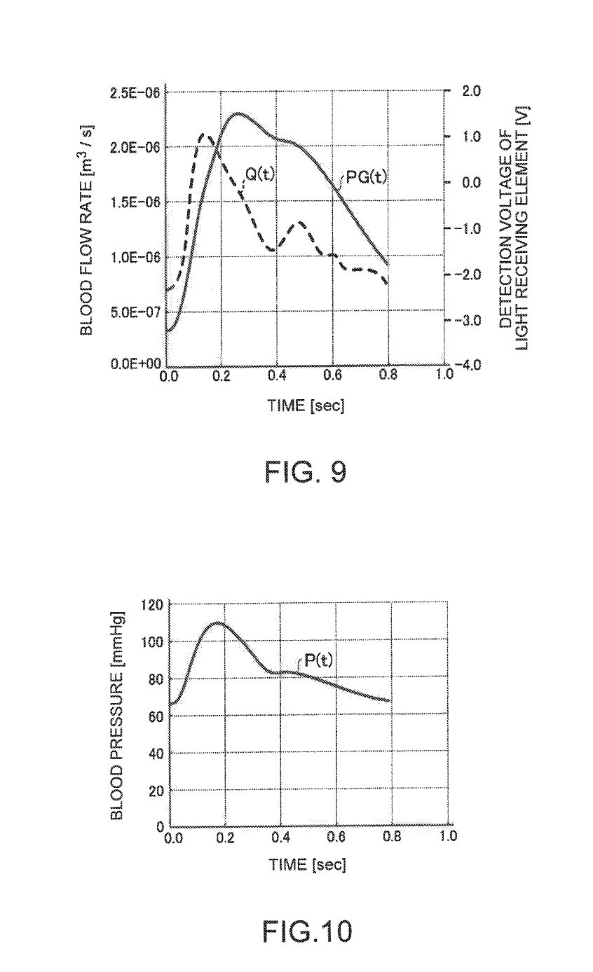

The change over time in the full power <I²> of the received light signal S1 obtained in Step S25 is equivalent to a plethysmogram of the wrist of the subject 100. If the values of the full power <I²> of the received light signal S1 calculated for each section are sequentially plotted, a waveform of a plethysmogram PG(t) illustrated in FIG. 9 is generated. The blood flow waveform Q(t) illustrated in FIG. 9 represents a graph of the change over time in the blood flow rate Q obtained in Step S24. The plethysmogram PG(t) and the blood flow waveform Q(t) illustrated in FIG. 9 are equivalent to approximately one beat of a pulse wave.

Hereinafter, a step of obtaining a change over time in the blood vessel cross-sectional area A will be described. The computational unit 422 calculates a blood vessel diameter d for each divided section, for which fast Fourier transform is performed, from Expression 6 using Lambert Beer's law, and calculates the blood vessel cross-sectional area A by substituting the blood vessel diameter d into Expression 7. Accordingly, the blood vessel cross-sectional area A is calculated every 20 milliseconds. As a result, a change over time in the blood vessel cross-sectional area A is obtained (Step S26).

$$2 d = \frac{1}{k} log ( \frac{I}{{I}_{0}} ) \qquad [ Expression 6 ]$$

k represents an absorption coefficient of blood, and I₀ represents intensities (irradiation intensities) of laser beams irradiated by the laser beam emitting unit 510.

$$A = \frac{1}{2} d \times \frac{1}{2} d \times π \qquad [ Expression 7 ]$$

Also, in the embodiment, a calculation period for the blood flow rate Q or the blood vessel cross-sectional area A is not limited to 20 milliseconds, and if the calculation period for the blood flow rate Q or the blood vessel cross-sectional area A is sufficiently smaller than that of one beat of a pulse wave, the calculation period can be determined to be an arbitrary length of time.

Steps S27 to S30 thereafter are the same as Steps S6 to S9 described in the first embodiment. That is, the computational unit 422 obtains the pulse wave propagation velocity PWV from Expression 3 described in the first embodiment, using the change over time in the blood flow rate Q obtained in Step S24 and the change over time in the blood vessel cross-sectional area A obtained in Step S26 (Step S27). The computational unit 422 obtains a blood pressure using Expression 4 described in the first embodiment (Step S28). FIG. 10 illustrates an example of a waveform of blood pressure P(t). The waveform of the blood pressure P(t) illustrated in FIG. 10 is equivalent to approximately one beat of a pulse wave.

The computational unit 422 determines the degree of arteriosclerosis using the pulse wave propagation velocity PWV obtained in Step S27 (Step S29). Thereafter, the control unit 40 outputs the pulse wave propagation velocity PWV, the blood pressure, and the degree of arteriosclerosis, which are obtained by the computational unit 422, to the display unit 60 together with a command instructing display (Step S30), and ends the biological information measurement process. The display unit 60 may display waveforms of the plethysmogram PG(t), the blood flow waveform Q(t), and the blood pressure P(t), and the like.

As described above, in the embodiment, it is possible to measure a plethysmogram as biological information regarding the subject 100 in addition to obtaining the same effects as in the first embodiment. That is, the biological information measurement apparatus 2 of the second embodiment is capable of measuring a plethysmogram in addition to a pulse wave propagation velocity, a blood pressure, and the degree of arteriosclerosis via measurement by an LDF method using laser beams. One type of the optical sensor 50 (the laser beam emitting unit 510 and the laser beam receiving unit 520) is capable of simultaneously measuring the biological information items.

### Third Embodiment

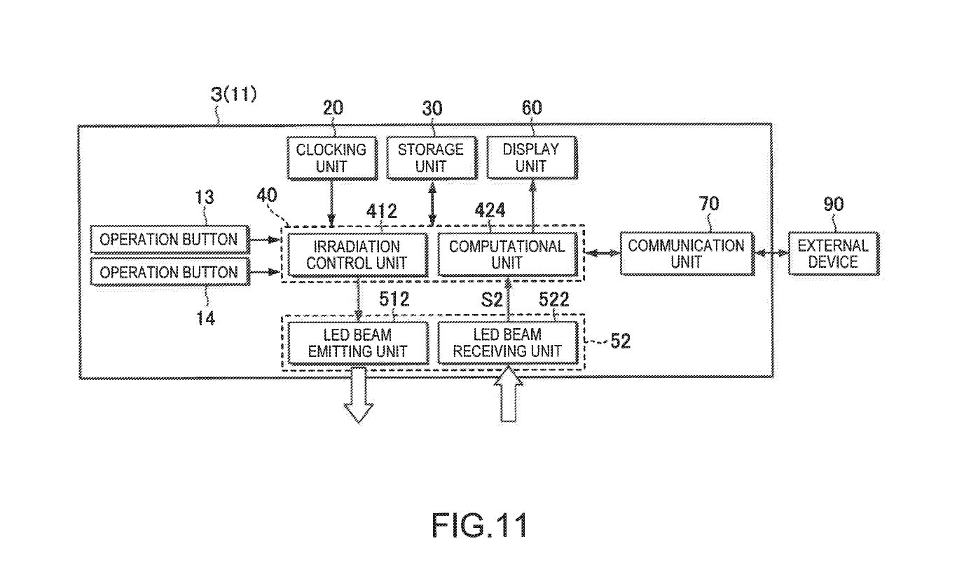

FIG. 11 is a block diagram illustrating the inner configuration of a biological information measurement apparatus 3 of a third embodiment of the invention. Also, in the embodiment, the reference signs used in the first embodiment are assigned to elements common to the first embodiment, and description thereof will be suitably omitted. The biological information measurement apparatus 3 of the third embodiment measures biological information regarding the subject 100 using light emitting diode (LED) beams instead of laser beams. The differences between the biological information measurement apparatus 3 illustrated in FIG. 11 and the biological information measurement apparatus 1 illustrated in FIG. 4 are that the biological information measurement apparatus 3 includes an irradiation control unit 412; an optical sensor 52 (an LED beam emitting unit 512 and an LED beam receiving unit 522); the received light signal S2; and a computational unit 424.

The irradiation control unit 412 controls irradiation of LED beams by the LED beam emitting unit 512. The LED beam emitting unit 512 includes an LED, and is controlled by the irradiation control unit 412 such that the LED beam emitting unit 512 irradiates the wrist of the subject 100 with LED beams which are an example of a measurement wave. LED beams irradiated by the LED beam emitting unit 512 are beams which are incoherent in a wider band compared to laser beams described in the first embodiment, and an example of non-laser beams. For example, LED beams irradiated by the LED beam emitting unit 512 have a wavelength of 535 nm.

The LED beam receiving unit 522 includes a light receiving element such as a photo diode; an amplifier; an A-to-D converter; and the like. The light receiving element has band-pass characteristics corresponding to the wavelength of LED beams irradiated by the LED beam emitting unit 512, selectively transmits only light of the corresponding wavelength region, and blocks light of other wavelength regions. The LED beam receiving unit 522 receives LED beams having passed through the living body of the subject 100 via the light receiving element, and generates and outputs the received light signal S2, which indicates a change over time in light receiving intensity of the LED beams, to the computational unit 424. The computational unit 424 obtains biological information regarding the subject 100 by computing the received light signal S2 output from the LED beam receiving unit 522.

After LED beams irradiated by the LED beam emitting unit 512 transmit through an epidermis, and are incident into the living body of the subject 100, the LED beams spreads through biological tissues while being repeatedly scattered and reflected, and a portion of the LED beams reaches the LED beam receiving unit 522, and is received by the light receiving element. A portion of the LED beams incident into the living body is absorbed by blood cells (mainly, hemoglobins) flowing through the blood vessel 110. Since the amount of blood cells inside of the blood vessel 110 during expansion of the blood vessel 110 is different from that during contraction thereof, and thus, the amplitude of the received light signal S2 generated by the LED beam receiving unit 522 varies periodically in correspondence with pulsations of the blood vessel 110.

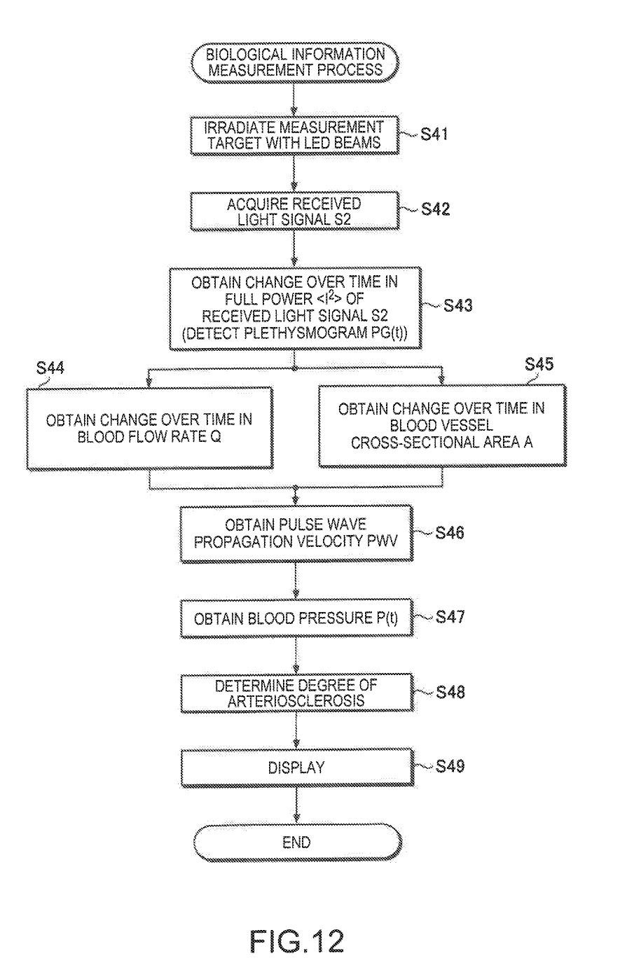

FIG. 12 is a flowchart illustrating a biological information measurement process of the third embodiment. The execution of the process illustrated in FIG. 12 is triggered by the control unit 40 in the same manner as in the process of the first embodiment illustrated in FIG. 6. If the process illustrated in FIG. 12 is started, first, the irradiation control unit 412 of the control unit 40 controls the LED beam emitting unit 512 such that irradiation of LED beams is started (Step S41). Accordingly, the wrist of the subject 100 is irradiated with LED beams, and the LED beam receiving unit 522 receives LED beams having passed through the inside of the living body of the subject 100, and outputs the received light signal S2 in correspondence with the received LED beams. The computational unit 424 of the control unit 40 acquires the received light signal S2 output from the LED beam receiving unit 522 (Step S42).

Subsequently, the computational unit 424 divides the acquired received light signal S2 into multiple sections having a predetermined length of time, for example, 20 milliseconds. The computational unit 424 calculates the full power <I²> of the received light signal S2 for each divided section from Expression 5 described in the second embodiment. Accordingly, the full power <I²> of the received light signal S2 is calculated every 20 milliseconds. As a result, a change over time in the full power <I²> of the received light signal S2 is obtained (Step S43). The change over time in the full power <I²> of the received light signal S2 is equivalent to a plethysmogram. If the values of the full power <I²> of the received light signal 82 calculated for each section are sequentially plotted, a waveform of the plethysmogram PG(t) illustrated in FIG. 9 is generated.

The change over time in the full power <I²> of the received light signal S2 obtained in Step S43 is equivalent to a change over time in volume V of blood. Accordingly, the computational unit 424 obtains a change over time in the blood flow rate Q from Expression 8 using the change (change V(t)_over time in the volume V of blood) over time in the full power <I²> of the received light signal S2 obtained in Step S43 (Step S44). That is, the computational unit 424 calculates the blood flow rate Q [m³/s], which is a volume velocity, every 20 milliseconds by time-differentiating the volume V [m³] of blood.

$$Q = \frac{dV ( t )}{dt} \qquad [ Expression 8 ]$$

In parallel with Step S44, the computational unit 424 obtains a change over time in the blood vessel cross-sectional area A using Expression 6 and Expression 7 described in the second embodiment (Step S45). That is, the computational unit 424 calculates the blood vessel cross-sectional area A by calculating the blood vessel diameter d for each divided section every 20 milliseconds from Expression 6 using Lambert Beer's law, and substituting the blood vessel diameter d into Expression 7. Also, in the embodiment, a calculation period for the blood flow rate Q or the blood vessel cross-sectional area A is not limited to 20 milliseconds, and if the calculation period for the blood flow rate Q or the blood vessel cross-sectional area A is sufficiently smaller than that of one beat of a pulse wave, the calculation period can be determined to be an arbitrary length of time.

Steps S46 to S49 thereafter are the same as Steps S6 to S9 described in the first embodiment. That is, the computational unit 424 obtains the pulse wave propagation velocity PWV from Expression 3 described in the first embodiment, using the change over time in the blood flow rate Q obtained in Step S44 and the change over time in the blood vessel cross-sectional area A obtained in Step S45 (Step S46). The computational unit 424 obtains a blood pressure using Expression 4 described in the first embodiment (Step S47).

The computational unit 424 determines the degree of arteriosclerosis using the pulse wave propagation velocity PWV obtained in Step S46 (Step S48). Thereafter, the control unit 40 outputs the pulse wave propagation velocity PWV, the blood pressure, and the degree of arteriosclerosis, which are obtained by the computational unit 424, to the display unit 60 together with a command instructing display (Step S49), and ends the biological information measurement process. Similar to the second embodiment, the display unit 60 may display waveforms of the plethysmogram PG(t), the blood flow waveform Q(t), and the blood pressure P(t), and the like.

As described above, the biological information measurement apparatus 3 of the embodiment also obtain the pulse wave propagation velocity PWV from a change over time in the blood flow rate Q and a change over time in the blood vessel cross-sectional area A. Therefore, it is not necessary for the subject 100 to wear the optical sensors 52 on multiple measurement sites such as a finger and a wrist, the number of measurement sites may be one, and the number of optical sensors 52 (the LED beam emitting unit 512 and the LED beam receiving unit 522) for measurement may be one. As a result, it is possible to reduce the size of the biological information measurement apparatus 3.

In the embodiment, the biological information measurement apparatus 3 is capable of obtaining both a change over time in the blood flow rate Q and a change over time in the blood vessel cross-sectional area A, which are required to obtain the pulse wave propagation velocity PWV, via measurement using LED beams. The biological information measurement apparatus 3 is capable of obtaining a blood pressure, the degree of arteriosclerosis, and a plethysmogram in addition to the pulse wave propagation velocity PWV as biological information regarding the subject 100, and is capable of continuously measuring the biological information items over a long period of time in a non-invasive and non-pressure manner. One type of the optical sensor 52 (the LED beam emitting unit 512 and the LED beam receiving unit 522) is capable of simultaneously measuring the biological information items.

### Fourth Embodiment

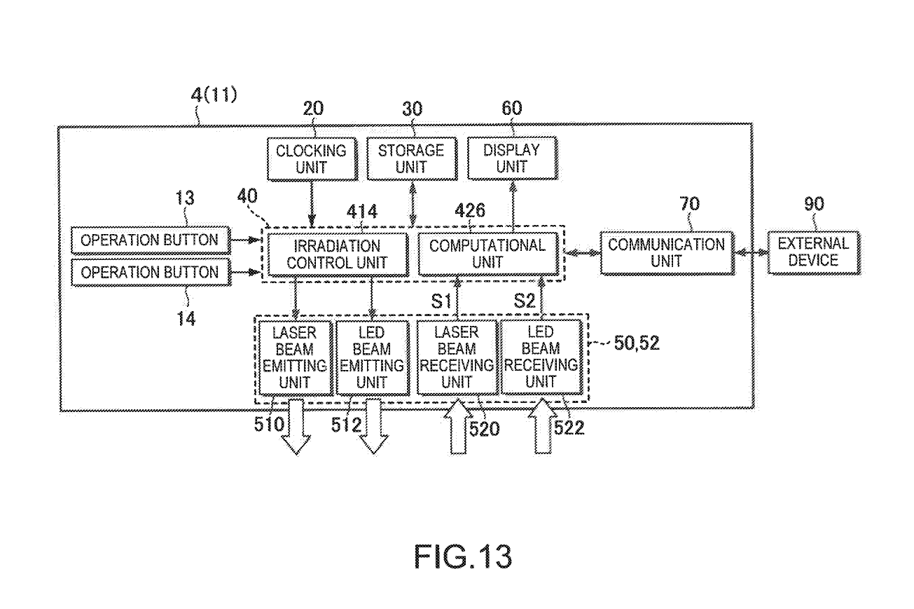

FIG. 13 is a block diagram illustrating the inner configuration of a biological information measurement apparatus 4 of a fourth embodiment of the invention. In the embodiment, the reference signs used in the first and third embodiments are assigned to elements common to the first and third embodiments, and description thereof will be suitably omitted. The biological information measurement apparatus 4 of the fourth embodiment measures biological information regarding the subject 100 using both laser beams and LED beams. The differences between the biological information measurement apparatus 4 illustrated in FIG. 13 and the biological information measurement apparatus 1 illustrated in FIG. 4 are that the biological information measurement apparatus 4 includes an irradiation control unit 414; the optical sensors 50 and 52 (the laser beam emitting unit 510, the laser beam receiving unit 520, the LED beam emitting unit 512 and the LED beam receiving unit 522); the received light signals S1 and S2; and a computational unit 426.

In FIG. 13, the optical sensor 50 includes the laser beam emitting unit 510 and the laser beam receiving unit 520, and the optical sensor 52 includes the LED beam emitting unit 512 and the LED beam receiving unit 522. In the embodiment, the optical sensor 50 (the laser beam emitting unit 510 and the laser beam receiving unit 520) is the same as the optical sensor 50 (the laser beam emitting unit 510 and the laser beam receiving unit 520) described in the first embodiment. The optical sensor 52 (the LED beam emitting unit 512 and the LED beam receiving unit 522) is the same as the optical sensor 52 (the LED beam emitting unit 512 and the LED beam receiving unit 522) described in the third embodiment.

The laser beam emitting unit 510 is an example of a first irradiation unit, and is the same as the laser beam emitting unit 510 described in the first embodiment. The laser beam emitting unit 510 is controlled by the irradiation control unit 414 such that the laser beam emitting unit 510 irradiates the wrist of the subject 100 with laser beams. The laser beam receiving unit 520 is an example of a first detection unit, and is the same as the laser beam receiving unit 520 described in the first embodiment. The laser beam receiving unit 520 receives laser beams having passed through the inside of the living body of the subject 100, and generates and outputs the received light signal (optical beat signal) S1, which indicates changes over time in light receiving intensity and frequency of laser beams, to the computational unit 426.

The LED beam emitting unit 512 is an example of a second irradiation unit, and is the same as the LED beam emitting unit 512 described in the third embodiment. The LED beam emitting unit 512 is controlled by the irradiation control unit 414 such that the LED beam emitting unit 512 irradiates the wrist of the subject 100 with LED beams. The LED beam receiving unit 522 is an example of a second detection unit, and is the same as the LED beam receiving unit 522 described in the third embodiment. The LED beam receiving unit 522 receives LED beams having passed through the inside of the living body of the subject 100, and generates and outputs the received light signal S2, which indicates a change over time in light receiving intensity of LED beams, to the computational unit 426.

The irradiation control unit 414 controls irradiation of laser beams performed by the laser beam emitting unit 510 and irradiation of LED beams performed by the LED beam emitting unit 512. The computational unit 426 obtains biological information regarding the subject 100 by computing the received light signal S1 output from the laser beam receiving unit 520 and the received light signal S2 output from the LED beam receiving unit 522.

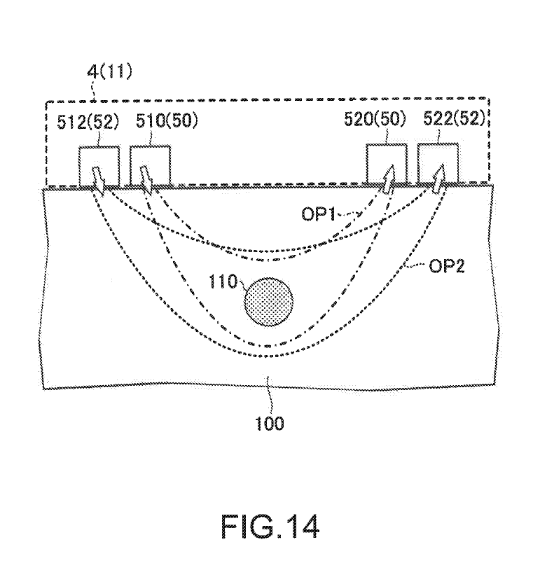

FIG. 14 illustrates the disposition of the optical sensors 50 and 52. If a high distribution frequency region of propagation paths of laser beams having reached the laser beam receiving unit 520 is schematically illustrated, the high distribution frequency region has a banana shape (OP1) illustrated by alternate long and short dash lines in FIG. 14. Similarly, if a high distribution frequency region of propagation paths of LED beams having reached the LED beam receiving unit 522 is schematically illustrated, the high distribution frequency region has a banana shape (OP2) illustrated by dotted lines in FIG. 14. A portion in the vicinity of the center of the laser beam passing region OP1 which has the widest width in the depth direction overlaps a portion in the vicinity of the center of the LED beam passing region OP2 which has the widest width in the depth direction. The laser beam emitting unit 510, the laser beam receiving unit 520, the LED beam emitting unit 512, and the LED beam receiving unit 522 are positioned such that the blood vessel 110 which is a measurement target is placed in a region in which both the portions overlap each other.

The passing regions OP1 and OP2 illustrated in FIG. 14 are mere images for the sake of convenience. Actual propagation paths of the laser beams having reached the laser beam receiving unit 520 are not limited to the passing region OP1 illustrated in FIG. 14, and various paths can be obtained. Similarly, actual propagation paths of the LED beams having reached the LED beam receiving unit 522 are not limited to the passing region OP2 illustrated in FIG. 14, and various paths can be obtained. In FIG. 14, for the sake of convenience, only one blood vessel 110 is illustrated, and actually, measurement targets are all blood vessels which are present on the propagation paths of the laser beams having reached the laser beam receiving unit 520 or the propagation paths of the LED beams having reached the LED beam receiving unit 522.

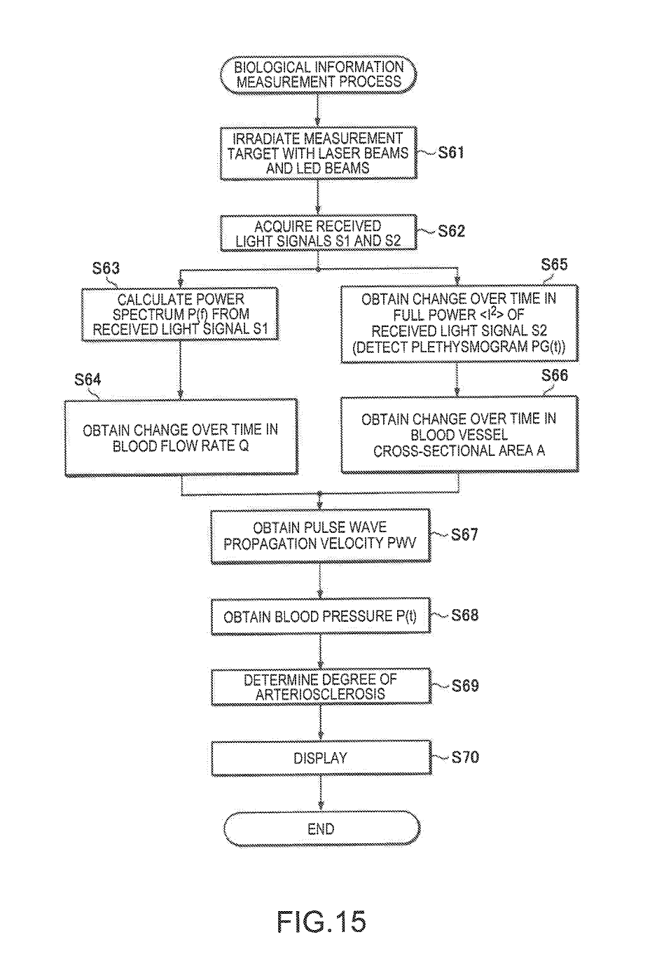

FIG. 15 is a flowchart illustrating a biological information measurement process of the fourth embodiment. The execution of the process illustrated in FIG. 15 is triggered by the control unit 40 in the same manner as in the process of the first embodiment illustrated in FIG. 6. If the process illustrated in FIG. 15 is started, first, the irradiation control unit 414 of the control unit 40 controls the laser beam emitting unit 510 such that irradiation of laser beams is started, and controls the LED beam emitting unit 512 such that irradiation of LED beams is started (Step S61). Accordingly, the wrist of the subject 100 is irradiated with laser beams and LED beams. The laser beam receiving unit 520 receives laser beams having passed through the inside of the living body of the subject 100, and outputs the received light signal S1 in correspondence with the received laser beams. The LED beam receiving unit 522 receives LED beams having passed through the inside of the living body of the subject 100, and outputs the received light signal S2 in correspondence with the received LED beams. The computational unit 426 of the control unit 40 acquires the received light signal S1 output from the laser beam receiving unit 520 and the received light signal S2 output from the LED beam receiving unit 522 (Step S62).

Subsequently, the computational unit 426 calculates the power spectrum P(f) by performing a frequency analysis process on the acquired received light signal (optical beat signal) S1 via fast Fourier transform (Step S63). The computational unit 426 obtains a change over time in the blood flow rate Q from Expression 1 described in the first embodiment, using the calculated power spectrum P(f) (Step S64). Steps S63 and S64 are the same as Steps S3 and S4 described in the first embodiment.

In parallel with Steps S63 and S64, the computational unit 426 calculates the full power <I²> of the received light signal S2 every predetermined periods, for example, every 20 milliseconds using Expression 5 described in the second embodiment, and obtains a change over time in the full power <I²> of the received light signal S2 (Step S65). The computational unit 426 calculates the blood vessel cross-sectional area A every predetermined periods, for example, every 20 milliseconds using Expression 6 and Expression 7 described in the second embodiment, and obtains a change over time in the blood vessel cross-sectional area A (Step S66). Steps S65 and S66 are the same as Steps S43 and S45 described in the third embodiment.

In the embodiment, a change over time in the blood flow rate Q is obtained via measurement by an LDF method using laser beams, and a change over time in the blood vessel cross-sectional area A is obtained from the measurement of a plethysmogram using LED beams. Also, in the embodiment, a calculation period for the blood flow rate Q or the blood vessel cross-sectional area A is not limited to 20 milliseconds, and if the calculation period for the blood flow rate Q or the blood vessel cross-sectional area A is sufficiently smaller than that of one beat of a pulse wave, the calculation period can be determined to be an arbitrary length of time.

Steps S67 to S70 thereafter are the same as Steps S6 to S9 described in the first embodiment. That is, the computational unit 426 obtains the pulse wave propagation velocity PWV from Expression 3 described in the first embodiment, using the change over time in the blood flow rate Q obtained in Step S64 and the change over time in the blood vessel cross-sectional area A obtained in Step S66 (Step S67). The computational unit 426 obtains a blood pressure using Expression 4 described in the first embodiment (Step S68).

The computational unit 426 determines the degree of arteriosclerosis using the pulse wave propagation velocity PWV obtained in Step S67 (Step S69). Thereafter, the control unit 40 outputs the pulse wave propagation velocity PWV, the blood pressure, and the degree of arteriosclerosis, which are obtained by the computational unit 426, to the display unit 60 together with a command instructing display (Step S70), and ends the biological information measurement process. Similar to the second embodiment, the display unit 60 may display waveforms of the plethysmogram PG(t), the blood flow waveform Q(t), and the blood pressure P(t), and the like.

As described above, in the embodiment, the biological information measurement apparatus 4 obtains a change over time in the blood flow rate Q via measurement by an LDF method using laser beams, and obtains a change over time in the blood vessel cross-sectional area A from the measurement of a plethysmogram using LED beams. It is possible to more accurately obtain a change over time in the blood flow rate Q via measurement by an LDF method using laser beams in comparison with that in a case where a change over time in the blood flow rate Q is obtained from the measurement of a plethysmogram using LED beams. In contrast, it is possible to more accurately obtain a change over time in the blood vessel cross-sectional area A from the measurement of a plethysmogram using LED beams in comparison with that in a case where a change over time in the blood vessel cross-sectional area A is obtained via measurement by an LDF method using laser beams.

Accordingly, in the embodiment, two types of the optical sensors 50 and 52 are required, and in contrast, it is possible to more accurately obtain a change over time in the blood flow rate Q and a change over time in the blood vessel cross-sectional area A in comparison with that in a case where the biological information measurement apparatuses 1 to 3 of the first to third embodiments obtain a change over time in the blood flow rate Q and a change over time in the blood vessel cross-sectional area A. As a result, it is possible to improve the accuracy of calculation of the pulse wave propagation velocity PWV.

In the embodiment, it is possible to obtain the pulse wave propagation velocity PWV using a change over time in the blood flow rate Q and a change over time in the blood vessel cross-sectional area A which are obtained from the same site (wrist). As a result, it is possible to more accurately obtain the pulse wave propagation velocity PWV of a local site (wrist). Since a site from which a change over time in the blood flow rate Q is measured by irradiating the site with laser beams is the same as a site from which a change over time in the blood vessel cross-sectional area A is measured by irradiating the site with LED beams, it is possible to further reduce the size of the biological information measurement apparatus 4 in comparison with that in a case where both the sites are not the same.

### Modification Example

The embodiments exemplarily illustrated above can be modified in various forms. Hereinafter, specific modification forms will be exemplified. Two or more forms arbitrarily selected from the following examples can be suitably combined together insofar as the two or more forms do not contract each other.

(1) If description is given with reference to the first embodiment, as illustrated in FIG. 1, a subject may wear the biological information measurement apparatus 1 on a wrist such that the main body portion 11 is positioned on a palm side, or such that the main body portion 11 is positioned on a back side of the hand. One of or both the laser beam emitting unit 510 and the laser beam receiving unit 520 may be provided on an inner peripheral surface of the belt 12 instead of being provided in the main body portion 11. The biological information measurement apparatus 1 may be a wearable device which can be mounted on a belt of an existing wrist watch. This modification can be applied to the biological information measurement apparatuses 2 to 4 described in the second to fourth embodiments.

(2) Each of the biological information measurement apparatuses 1 to 4 includes a small reader/writer as a storage medium such as a memory card, and may be configured to exchange data with the external device 90 via the storage medium.

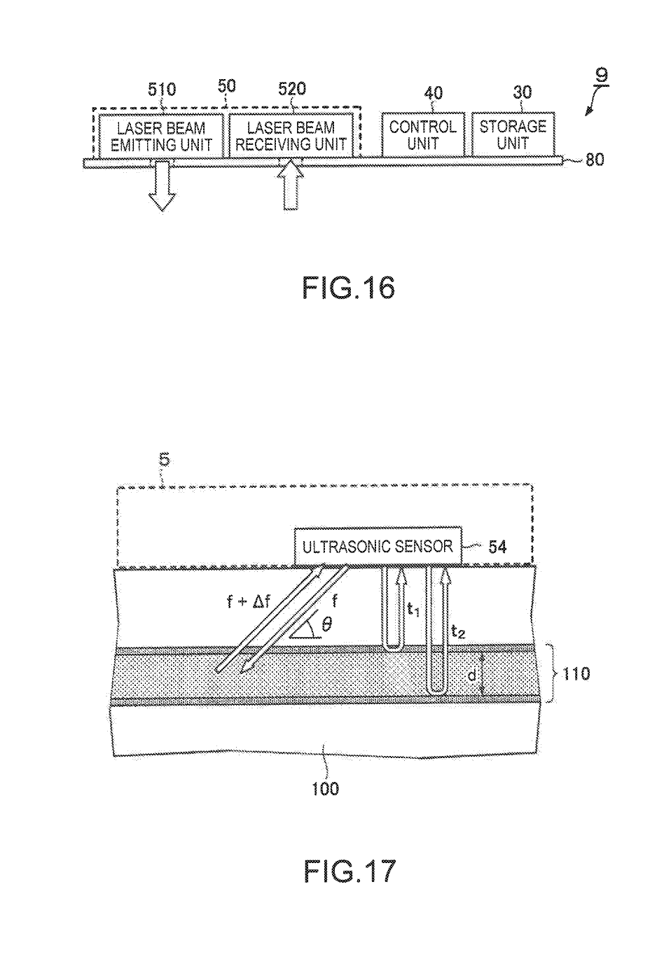

(3) If description is give with reference to the first embodiment, the biological information measurement apparatus 1 (refer to FIG. 4) may not necessarily include the operation buttons 13 and 14, the clocking unit 20, and the communication unit 70 as configuration elements. The biological information measurement apparatus 1 may be configured to output the results of measuring a pulse wave propagation velocity, a blood pressure, the degree of arteriosclerosis, and the like to the external device 90 via the communication unit 70. In this case, the biological information measurement apparatus 1 is not necessarily provided with the display unit 60. As illustrated in FIG. 16, a biological information measurement apparatus may be a biological information measurement module 9 having a configuration in which the optical sensor 50 (the laser beam emitting unit 510 and the laser beam receiving unit 520), the control unit 40, and the storage unit 30 are mounted on a substrate (for example, circuit board) 80. The measurement module 9 may be assembled into an existing wearable device such as a wrist watch. In this case, the biological information measurement module (biological information measurement apparatus) 9 does not require a housing of the main body portion 11 and the belt 12 as configuration elements. Such a deformation can be applied to the biological information measurement apparatuses 2 to 4 described in the second to fourth embodiments.

(4) In the fourth embodiment, desirably, a site from which a change over time in the blood flow rate Q is measured by irradiating the site with laser beams is basically the same as a site from which a change over time in the blood vessel cross-sectional area A is measured by irradiating the site with LED beams. In contrast, both sites are not necessarily limited to the same site. Alternatively, both the sites may be different sites such as a palm side and a hand back side of a wrist.

(5) The biological information measurement apparatus 4 of the fourth embodiment may include one light receiving unit including a single light receiving element which receives both laser beams irradiated by the laser beam emitting unit 510 and LED beams irradiated by the LED beam emitting unit 512, instead of including the laser beam receiving unit 520 and the LED beam receiving unit 522 as separate elements. In this case, the light receiving element of the light receiving unit has band-pass characteristics corresponding to both a wavelength of laser beams irradiated by the laser beam emitting unit 510 and a wavelength of LED beams irradiated by the LED beam emitting unit 512. The light receiving unit generates the received light signal (optical beat signal) S1 which represents changes over time in light receiving intensity and frequency of laser beams having passed through the inside of the living body of the subject 100, and the received light signal S2 which represents a change over time in light receiving intensity of LED beams having passed through the inside of the living body of the subject 100. In this configuration, the number of light receiving units may be one, and it is not necessary to separately provide a light receiving unit for receiving laser beams and a light receiving unit for receiving LED beams. As a result, it is possible to further simplify the configuration of the biological information measurement apparatus and to further reduce the size of the biological information measurement apparatus than those of the biological information measurement apparatus 4 of the fourth embodiment.

(6) A site which is a measurement target is not limited to a wrist, and may be a finger, an arm, a leg, or a head. Accordingly, the biological information measurement apparatuses 1 to 4 are not limited to a wrist watch type, and alternatively, may be a wearable device which the subject 100 can wear on a measurement target site of the body. For example, each of the biological information measurement apparatuses 1 to 4 may be a smart phone that is fixed to an upper arm of the subject 100 with a belt. A biological information measurement apparatus according to the invention is not limited to a wearable device. The invention may be applied to a stationary blood pressure meter used in a medical institution. In this case, measurement is performed in a state where a probe is brought into contact with a measurement target site.

(7) The wavelength of laser beams or LED beams is not limited to the wavelengths exemplified in the embodiments. It is possible to suitably determine a wavelength while taking into consideration propagation characteristics of laser beams or LED beams inside of a living body, or the extent that laser beams or LED beams are absorbed by blood. Super luminescent diode (SLD) beams may be used instead of LED beams, and non-LED beams are not limited to LED beams.

(8) A measurement wave with which a living body is irradiated is not limited to laser beams or LED beams, and alternatively, may be a sound wave such as an ultrasonic wave. FIG. 17 is a schematic view illustrating a principle of measuring biological information via an ultrasonic sensor 54. A biological information measurement apparatus 5 of this modification example includes the ultrasonic sensor 54 instead of an optical sensor. The ultrasonic sensor 54 includes an irradiation unit which irradiates the subject (living body) 100 with ultrasonic waves which are an example of a measurement wave, and a detection unit which detects ultrasonic waves having being reflected from the inside of the living body.

If the frequency of ultrasonic waves (irradiation waves) with which the blood vessel 110 is irradiated by the irradiation unit of the ultrasonic sensor 54 is assumed to be f, ultrasonic waves (reflected waves) reflected by blood cells such as red blood cells flowing through the blood vessel 110 are subjected to a Doppler shift corresponding to the flow velocity of the blood cells, and the frequency of the ultrasonic waves (reflected waves) is changed to f+Δf. Accordingly, similar to measurement by an LDF method using laser beams, the biological information measurement apparatus 5 is capable of obtaining a change over time in the blood flow rate Q by measuring a frequency change Δf of the reflected waves with respect to that of the irradiation waves.

The biological information measurement apparatus 5 is capable of obtaining the blood vessel cross-sectional area A by measuring the blood vessel diameter d from a time difference Δt (t₂−t₁) between a time t₁ when ultrasonic waves have been reflected by an epidermis side wall of the blood vessel 110 and reflected waves have reached the biological information measurement apparatus 5 and a time t₂ when ultrasonic waves have been reflected by a wall of the blood vessel 110 opposite to the epidermis and reflected waves have reached the biological information measurement apparatus 5, and by substituting the value of the measured blood vessel diameter d into Expression 7. Accordingly, the biological information measurement apparatus 5 is capable of obtaining a change over time in the blood vessel cross-sectional area A by calculating the blood vessel cross-sectional area A every predetermined periods, for example, every 20 milliseconds.

As described above, the biological information measurement apparatus 5 including the ultrasonic sensor 54 instead of an optical sensor is capable of obtaining the pulse wave propagation velocity PWV using a change over time in the blood flow rate Q and a change over time in the blood vessel cross-sectional area A from Expression 3. After obtaining the pulse wave propagation velocity PWV, the biological information measurement apparatus 5 is capable of obtaining the blood pressure P(t) using Expression 4, or determining the degree of arteriosclerosis from the value of the pulse wave propagation velocity PWV.

If sound waves such as ultrasonic waves are used as measurement waves, it is possible to obtain a change over time in the blood vessel cross-sectional area A from the time difference Δt (t2−t1) between times when two reflected waves reflected by a wrist side wall and a deep side wall of the blood vessel 110 have reached the biological information measurement apparatus 5. Accordingly, the blood vessel 110 which is a measurement target is limited to a blood vessel having a certain degree of thickness. Since the blood vessel 110 which is a measurement target is limited by a thickness, the degree of freedom in installing the ultrasonic sensor 54 is low.

In contrast, if laser beams or LED beams are used as measurement waves as described in the aforementioned embodiments, a change over time in the blood vessel cross-sectional area A is obtained using properties of blood absorbing a portion of irradiated beams. Accordingly, the blood vessel 110 which is a measurement target is not limited to a blood vessel having a certain degree of thickness. That is, the blood vessel 110 which is a measurement target may be a blood vessel narrower than that of a blood vessel in a case where sound waves are used as measurement waves. The number of blood vessels serving as candidates of measurement targets is greater than that in a case where sound waves are used as measurement waves. As a result, the degree of freedom in installing the optical sensors 50 and 52 is high.

Particularly, in such a wearable biological information measurement apparatus, using light as measurement waves rather than sound waves is advantageous in that there is no limitation to the thickness of the blood vessel 110 which is a measurement target, or the degree of freedom in installing a sensor is high. An optical sensor is advantageous in that the optical sensor has a size smaller than that of an ultrasonic sensor.

(9) A biological information measurement apparatus may be configured to measure only a pulse wave propagation velocity as biological information. The biological information measurement apparatus may be configured to measure one or more of a blood pressure, the degree of arteriosclerosis, and a plethysmogram in addition to a pulse wave propagation velocity. The biological information measurement apparatus may be configured to measure a pulse rate, a blood flow velocity, or the like in addition to the biological information items.

(10) A biological information measurement apparatus is not limited to a reflective type apparatus in which an irradiation unit and a detection unit are disposed side by side, and which detects measurement waves reflected from a measurement site. A biological information measurement apparatus may be a transmitting type apparatus in which a detection unit is provided to face an irradiation unit with a measurement site such as a fingertip interposed therebetween, and which detects measurement waves having transmitted through the measurement site.

(11) A blood vessel which is a measurement target may not be an artery but an arteriole. In this case, a blood vessel which is a measurement target is a site, the position of which is narrower than that of an artery. As a result, it is possible to reduce the separation distance between an irradiation unit and a detection unit, and to further reduce the size of a biological information measurement apparatus. A living body which is a measurement target may be an animal other than a human.

The entire disclosure of Japanese Patent Application No. 2016-042291 is hereby incorporated herein by reference.

What is claimed is:

1. A biological information measurement apparatus comprising:
an irradiation unit configured to irradiate a living body with light or sound waves as measurement waves;
a detection unit configured to detect the measurement waves having passed through the inside of the living body; and
a computational unit configured to obtain a change over time in blood flow rate and a change over time in blood vessel cross-sectional area based on a detection result from the detection unit, and to obtain a pulse wave propagation velocity from the change over time in blood flow rate or the change over time in blood vessel cross-sectional area.

2. The biological information measurement apparatus according to claim 1,
wherein the computational unit obtains a blood pressure or the degree of arteriosclerosis using the pulse wave propagation velocity.

3. The biological information measurement apparatus according to claim 1,
wherein the measurement waves are laser beams,
the detection unit generates an optical beat signal representing changes over time in light receiving intensity and frequency of the laser beams having passed through the inside of the living body, and
the computational unit obtains the change over time in blood flow rate and the change over time in blood vessel cross-sectional area from the optical beat signal generated by the detection unit.

4. The biological information measurement apparatus according to claim 3,
wherein the computational unit obtains a change over time in the full power of the optical beat signal.

5. The biological information measurement apparatus according to claim 1,
wherein the measurement waves are non-laser beams,
the detection unit generates a received light signal representing a change over time in light receiving intensity of the non-laser beams having passed through the inside of the living body, and
the computational unit obtains the change over time in blood flow rate and the change over time in blood vessel cross-sectional area from the received light signal generated by the detection unit.

6. The biological information measurement apparatus according to claim 1,
wherein the irradiation unit includes a first irradiation unit configured to irradiate the living body with laser beams, and a second irradiation unit configured to irradiate the living body with non-laser beams,
the detection unit includes a first detection unit configured to detect the laser beams having passed through the inside of the living body, and a second detection unit configured to detect the non-laser beams having passed through the inside of the living body, and
the computational unit obtains a change over time in blood flow rate based on a detection result from the first detection unit, and obtains a change over time in blood vessel cross-sectional area based on a detection result from the second detection unit.

7. The biological information measurement apparatus according to claim 1,
wherein the irradiation unit includes a first irradiation unit configured to irradiate the living body with laser beams, and a second irradiation unit configured to irradiate the living body with non-laser beams,
the detection unit detects the laser beams and the non-laser beams having passed through the inside of the living body, and
the computational unit obtains a change over time in blood flow rate based on a result of detecting the laser beams via the detection unit, and obtains a change over time in blood vessel cross-sectional area based on a result of detecting the non-laser beams via the detection unit.

8. The biological information measurement apparatus according to claim 6,
wherein a site of the living body, from which a change over time in blood flow rate is obtained by irradiating the site with the laser beams, is the same as a site of the living body from which a change over time in blood vessel cross-sectional area is obtained by irradiating the site with the non-laser beams.

9. A biological information measurement method comprising:
irradiating a living body with light or sound waves as measurement waves via a biological information measurement apparatus;
detecting the measurement waves, which have passed through the inside of the living body, via the biological information measurement apparatus;
obtaining a change over time in blood flow rate and a change over time in blood vessel cross-sectional area based on a detection result via the biological information measurement apparatus; and
obtaining a pulse wave propagation velocity from the change over time in blood flow rate and the change over time in blood vessel cross-sectional area.
